(19) United States
(12) Patent Application Publication
(10) Pub. No.: US 2016/0354162 A1
(43) Pub. Date: Dec. 8, 2016

(54) DRILLING CONTROL SYSTEM AND DRILLING CONTROL METHOD

(71) Applicant: NATIONAL TAIWAN UNIVERSITY, Taipei (TW)

(72) Inventors: PING-LANG YEN, Taipei (TW); Ting-Ya Hsiao, Taichung (TW); Chih-Min Yang, New Taipei (TW)

(21) Appl. No.: 15/169,656

(22) Filed: May 31, 2016

Related U.S. Application Data
(60) Provisional application No. 62/170,123, filed on Jun. 2, 2015.

(51) Int. Cl. A61B 34/20; A61B 17/17; A61B 17/16

(52) U.S. Cl. CPC A61B 34/20; A61B 17/1671; A61B 17/1703; A61B 2034/2072; A61B 2017/00119

(57) ABSTRACT

The present disclosure generally relates to the drilling control system and the drilling control method for surgical applications. The drilling control system may comprise a drilling device, a spatial sensor system and a control unit. The control unit may receive and store biomechanical information, mechanical information and spatial information to generate control output. With the present disclosure, the accuracy and safety of drilling process is greatly improved.

### FIELD

The subject matter herein generally relates to a drilling control system and a drilling control method.

### BACKGROUND

Tissue penetration is one of the important surgical procedures, such as soft tissue biopsy, lumbar puncture, bone marrow biopsy, craniotomy or osteotomy. Osteotomy is frequently performed in orthopedic surgery and neurosurgery. Usually, a bone drilling machine is used by a surgeon to make a hole for screw insertion in orthopedic surgery, such as internal fixation, external fixation, artificial joint replacement, spinal fusion, and spinal fixation. Implantation of pedicle screws is extremely risky because of the small target and the extreme closeness of neural tissue all around the pedicle of the vertebra, such as cervical, thoracic and lumbar spines. For example, performed in the posterior lumbar interbody fusion (PLIF).

Conventional surgery needs a complete pre-operative evaluation and planning to decide the drilling location and trajectory. However, with limited surgical incision, the surgeon may only recognize the drilling trajectory through surface anatomy and need to repeat fluoroscopic imaging to confirm the drilling trajectory. Not only has the problem of unnecessary doses of X-ray exposure to the surgeons and patients but also the inaccuracy of the procedure remained unsolved. Many image guided medical instruments assist surgeons by visualization of the location of the bone drilling machine. Though, the drilling process still greatly depends on the operator's experience to align the tool and the severe failure events are hardly detected by the surgeons before those events occur. The inaccuracy often leads irreversible damage to the patients in the certain critical surgical procedures.

Therefore, it would be very advantageous to provide surgeons a system or a method for controlling the drilling process precisely. With the present disclosure, the failure events occurred during drilling process is greatly reduced. It will be appreciated that the drilling control system and method assist the surgeon in accurate controlling the spindle speed and distinguishing among different tissue types.

### BRIEF DESCRIPTION OF THE DRAWINGS

Implementations of the present technology will now be described, by way of example only, with reference to the attached figures.

FIG. 1A shows the system diagram of the drilling control system.

FIG. 1B shows an example of the drilling control system coupled to a display module and a spatial sensor system when applied on spinal surgery.

FIG. 2A shows an information flow diagram that the control unit may receive biomechanical information and drilling information and may generate a control output.

FIG. 2B shows a diagram of calculation of the discrepancy according to the biomechanical and the drilling information.

FIG. 2C shows a flow diagram of the drilling control method.

FIG. 3A shows biomechanical information with the planned drilling trajectory visualized in three-dimensional model.

FIG. 3B shows the planned spindle speed along the planned drilling trajectory.

FIG. 3C shows biomechanical property along the drilling depth.

FIG. 4A shows biomechanical information simulated according to the force along z-axis as a function of drilling depth. FIG. 4B shows biomechanical information simulated according to the torque along z-axis as a function of drilling depth. FIG. 4C shows biomechanical information simulated according to the force along y-axis as a function of drilling depth. FIG. 4D shows biomechanical information simulated according to the torque along y-axis as a function of drilling depth. FIG. 4E shows biomechanical information simulated according to the force along x-axis as a function of drilling depth. FIG. 4F shows biomechanical information simulated according to the torque along x-axis as a function of drilling depth.

FIG. 5A shows one example of the drilling control system applied on spinal surgery.

FIG. 5B shows a graph illustrating the drilling information and the biomechanical information along the drilling depth.

FIG. 5C shows a graph illustrating the discrepancy index along the drilling depth.

FIG. 6A shows an example of the force/torque sensor coupled to the drilling motor.

FIG. 6B shows an example of the joint force sensor coupled to the kinetic pairs.

FIG. 6C shows an example of the motor current sensor coupled to the driving motor.

FIG. 6D shows an example of the robotic assembly comprising universal-prismatic-spherical joint pairs.

FIG. 6E shows an example of the robotic assembly comprising universal-prismatic-universal joint pairs.

FIG. 7A shows an example of the operation base, which is a fixation base.

FIG. 7B shows an example of the operation base, which is a combination of a fixation base and a handheld handle.

FIG. 7C shows an example of the operation base, which is a handheld handle.

FIG. 8A shows an example of the drilling control system coupled to the spatial sensor system, which is an optical tracking system.

FIG. 8B shows an example of the drilling control system coupled to the spatial sensor system, which comprises multiple inertial measurement units and a drilling trocar with a position sensor.

FIG. 8C shows an example of the drilling control system coupled to the spatial sensor system, which comprises multiple inertial measurement units and a drilling trocar with a proximeter.

FIG. 9 shows an example of the drilling control system coupled to a C-arm fluoroscopy.

FIG. 10 shows an example of the drilling control system capable of adjust alignment by the control unit.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

Several definitions that apply throughout this disclosure will now be presented.

The term “coupled” is defined as connected, whether directly or indirectly through intervening components, and is not necessarily limited to physical connections. The connection can be such that the objects are permanently connected or releasably connected. The term “comprising” means “including, but not necessarily limited to”; it specifically indicates open-ended inclusion or membership in a so-described combination, group, series and the like.

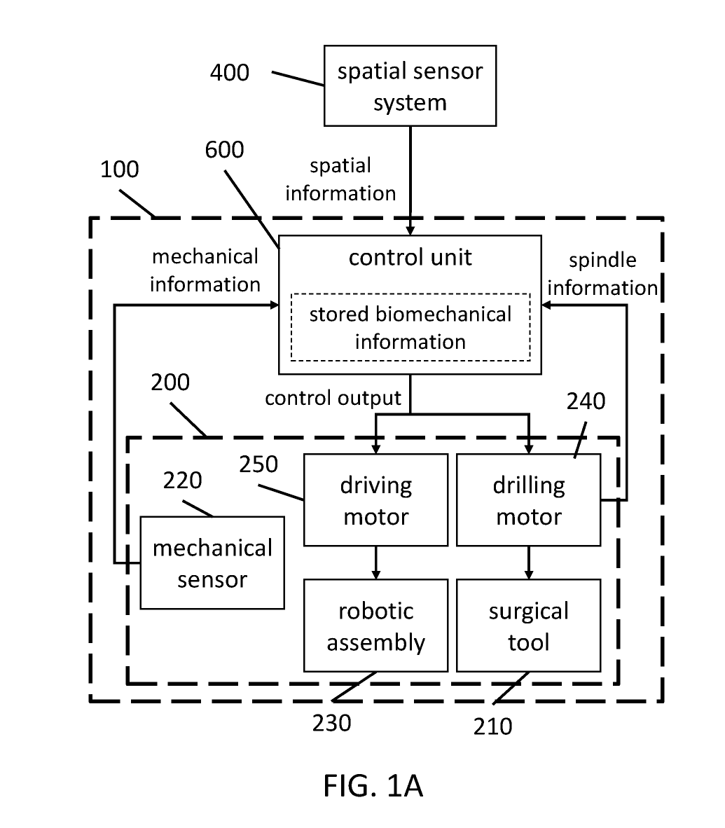

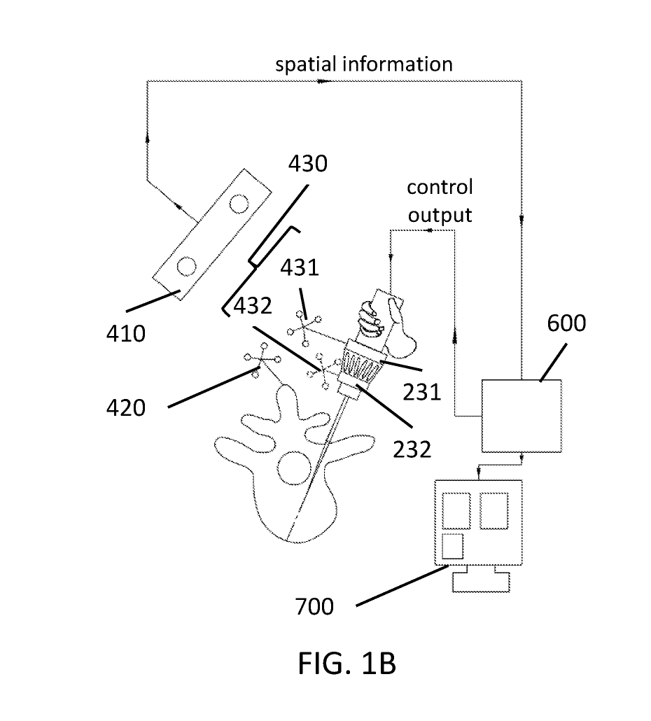

In one example as shown in FIG. 1A, a drilling control system may comprise a control unit 600 and a drilling device 200. The drilling control system 100 may be coupled to a spatial sensor system 400 to receive spatial information. The spatial sensor system 400 is configured to detect the spatial information of the drilling device 200 and the fiducial marker on the patient and to deliver the spatial information to the control unit 600. The control unit 600 is configured to receive and store control input, to calculate control output according to the control input and to deliver control output to the drilling device 200. The control input may comprise spatial information, mechanical information, spindle information and biomechanical information. The control unit 600 may receive control input from outside of the control unit 600, such as, the spatial sensor system 400, the drilling device 200, computed tomography (CT), magnetic resonance imaging (MRI), ultrasonography or a C-arm fluoroscopy or may store the control input, such as biomechanical information pre-processed from medical images. The drilling device 200 is configured to deliver mechanical information and spindle information to the control unit 600, to receive the control output from the control unit 600 and to perform drilling process according to the control output. The drilling device 200 may comprise a mechanical sensor 220, a drilling motor 240, a driving motor, a robotic assembly 230, and a surgical tool 210. The mechanical sensor 220 may detect the mechanical information and deliver the mechanical information as a part of the control input to the control unit 600. The control output may be delivered to the drilling motor 240 for controlling the spindle speed of the surgical tool 210 or to the robotic assembly 230 for controlling the orientation or the position of the surgical tool 210.

The drilling device 200 may comprise a surgical tool 210, a drilling motor 240 driving the surgical tool 210, a mechanical sensor 220 detecting mechanical information, a robotic arm assembly and an operation base 300 coupled to the robotic arm assembly. The surgical tool 210 is configured to create a hole powered by the drilling motor 240. The surgical tool 210 may be a drill bit. The drilling motor 240 provides rotational power to drive the surgical tool 210 and may be controlled by the control unit 600. The drilling motor may deliver the spindle information to the control unit according to the electric current passing through the drilling motor or via a motor rotation speed detection integrated circuit. In addition, the drilling motor may comprise a rotary encoder, a synchro, a resolver, a rotary variable differential transducer (RVDT), or rotary potentiometer to obtain the spindle speed of the surgical tools driven by the drilling motor and deliver the spindle information to the control unit. Usually, the drilling motor 240 is an electric motor such as a stepper motor, a servo motor or an ultrasonic motor. The servo motor may be an alternative current (AC) servo motor, a direct current (DC) (such as brush or brushless) servo motor. The mechanical sensor 220 is configured to detect mechanical information. The mechanical information may be the force or the torque applied on the surgical tool 210 and he force or the torque may be measured along x-axis, y-axis or z-axis. The mechanical sensor 220 may be a force sensor to detect the axial force or the deviation force. The mechanical sensor 220 may be a torque sensor to detect the rotational torque. The mechanical sensor 220 may be a torque sensor to detect the rotational torque. The robotic assembly 230 is configured to adjust the position and/or the orientation of the surgical tool 210. The robotic assembly 230 comprises at least a kinetic pair, such as a prismatic arm, a universal joint pair, a screw joint pair or a cylindrical joint pair. Also, the robotic assembly 230 may comprise multiple kinetic pairs, such as Stewart type robotic arm or delta robotic arm. Each kinetic arm may be powered by a driving motor controlled by the control unit 600. The operation base 300 is configured to serve as a static mechanical support to the robotic assembly 230 and to position the drilling device 200 near the surgical area. The operation base 300 may be a handheld handle 320, a fixation stand 310 or a combination of a handheld handle and a fixation stand. The handheld handle gripped by a surgeon provides mobility during drilling process. The fixation stand may be coupled to the operation table fixed on the ceiling or fixed on the floor so that a surgeon may save most effort for handling the drilling device 200.

The spatial sensor system 400 is configured to detect the spatial information of the drilling device 200 corresponding to the fiducial marker at a surgical area. The spatial sensor system 400 may be optical tracking system, magnetic tracking system, ultrasound tracking system, global positioning system (GPS), wireless positioning system, inertial measurement unit (IMU) device or visible light camera device for localization of the drilling device 200. For example, the spatial sensor system 400 may be an optical tracking system comprising a tracking sensor 410, a device marker 430 and a fiducial marker 420. The spatial information comprises three-dimensional coordinates and may further be recorded along with time series.

In one example as shown in FIG. 1B, the spatial sensor system 400 may be an optical tracking system comprising a tracking sensor 410, a fiducial marker 420 and a device marker 430. The fiducial marker 420 and the device marker 430 may comprise an array of tracking points arranged in a specific geometry, for example, triangular arrangement or quadrilateral arrangement, for precise recognition with the use of the tracking sensor 410. The fiducial marker 420 may be placed on the subject's skin surface or on a certain anatomical site, such as spinous process. The device marker 430 may be placed on the drilling device 200. For example, the spatial sensor system 400 may comprise two device markers, wherein the first device marker 431 is coupled to the base platform of the drilling device 200 and the second device marker 432 is coupled to the moving platform 232 of the drilling device 200. The tracking sensor 410 is capable of sensing the spatial information according to the relative location of the fiducial marker 420 and the device markers 430 so that the displacement and/or the orientation of the drilling device 200 can be recorded. The spatial information may comprise position and/or orientation in the sensing area, wherein the position in the area are noted as x, y, z and the orientation along x-axis, y-axis, z-axis are noted as α, β, γ. The drilling control system may further comprise a user interface 700 coupled to the control unit 600 to visualize the biomechanical information and the drilling information.

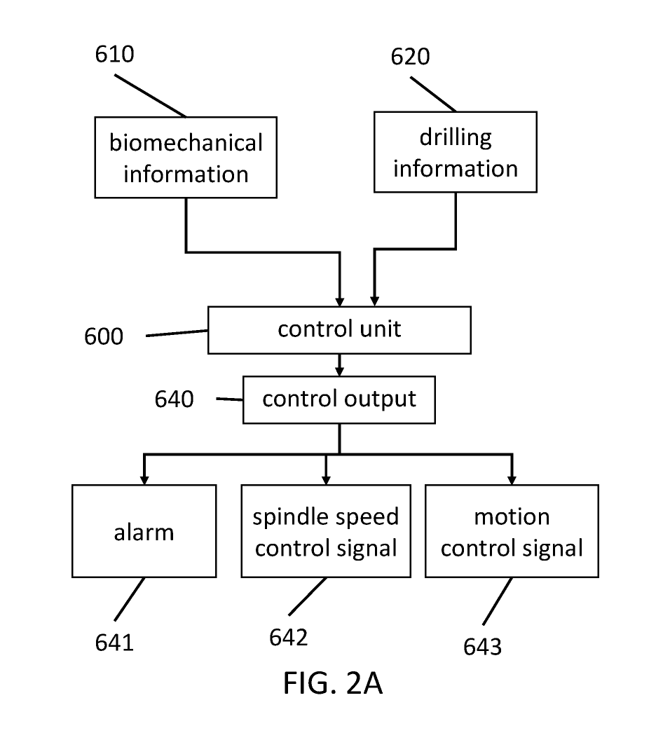

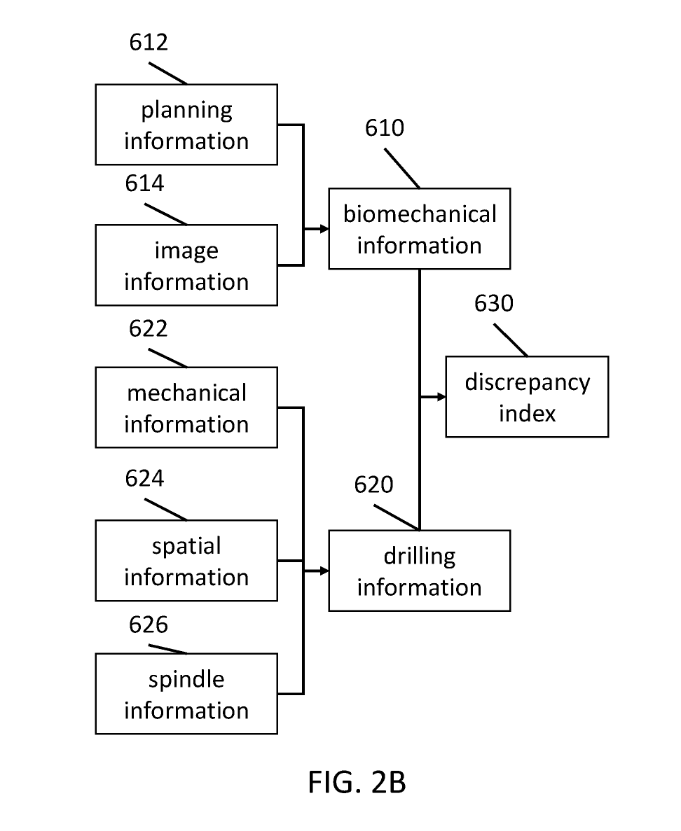

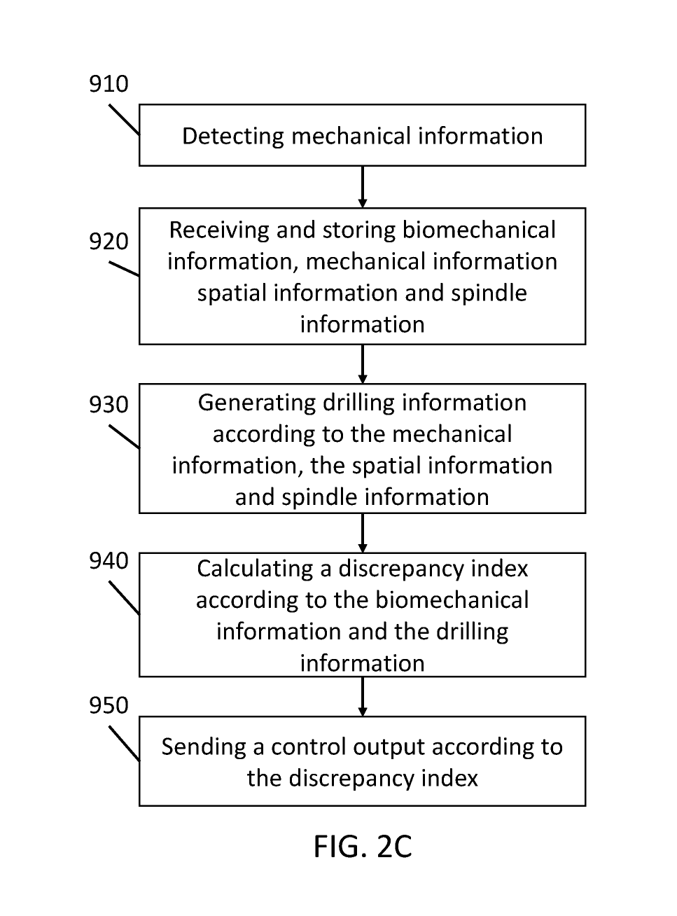

In one example as shown in FIG. 2A, the drilling control system is configured to generate control output 640 according to the received control input for controlling the drilling device 200 during the drilling process. The control input may comprise the biomechanical information 610 and the drilling information 620. The control unit 600 may send the control output to control the drilling device 200. For example, the control output may be a visual or audio alarm to alert the surgeon, may be a spindle speed control signal to the drilling motor 240, or may be a motion control signal to the robotic assembly 230.

As shown in FIG. 2B, the control unit calculate discrepancy index 630 according to the biomechanical information 610 and the drilling information 620. The biomechanical information may be generated by the control unit or other processing units according to the image information and the planning information. The biomechanical information 610 may be modeled from image information such as an X-ray image of the surgical area or from a series of computed chromatography (CT) images of the surgical area. For example, the image information may comprise three-dimensional voxels with CT numbers. The planning information may comprise the planned spindle speed at each voxel and may further comprise the planned feed rate. Therefore, the biomechanical properties of each voxel may be generated according to the planned information. The biomechanical information 610 may comprises one-dimensional coordinate with corresponding biomechanical properties, may comprise two-dimensional pixels with corresponding biomechanical properties or may comprise three-dimensional voxels with corresponding biomechanical properties. The biomechanical properties may represent stiffness, hardness, smoothness, drilling impedance or resistance. The drilling information 620 is generated by the control unit 600 according to the mechanical information 622, the spatial information 624 and the spindle information 626. The drilling information 620 may be generated from the mechanical information 622 as a function of the spatial information 624. The mechanical information 622 is the force or torque in specific direction detected by the mechanical sensor 220. The spatial information 624 comprises the location of the drilling device 200 corresponding to the anatomical site and may be used to calculate feed rate. The spindle information may comprise the spindle speed of the surgical tool or the drilling motor. The spindle information 626 may be delivered from the drilling motor to the control unit so that the control unit may confirm and adjust the spindle speed consistent with the planning information.

As shown in FIG. 2C, the drilling control method may be performed at a drilling control system. The drilling control method comprises detecting 910 mechanical information; receiving and storing 920 biomechanical information, mechanical information spatial information and spindle information; generating 930 drilling information according to the mechanical information, the spatial information, and spindle information; calculating 940 a discrepancy index according to the biomechanical information and the drilling information; sending 950 a control input according to the discrepancy index. In one example, the detecting step 910 is performed at a mechanical sensor of a drilling device in the drilling control system. The receiving and storing step 920 is performed at a control unit of the drilling control system wherein the biomechanical information may be received from a medical imaging device (such as CT or X-ray) or a medical image processing server, the mechanical information is received from the mechanical sensor, the spatial information is received from a spatial sensor system and the spindle information is received from a drilling motor. The generation step 930, the calculating step 940, and the sending step 950 is performed at the control input.

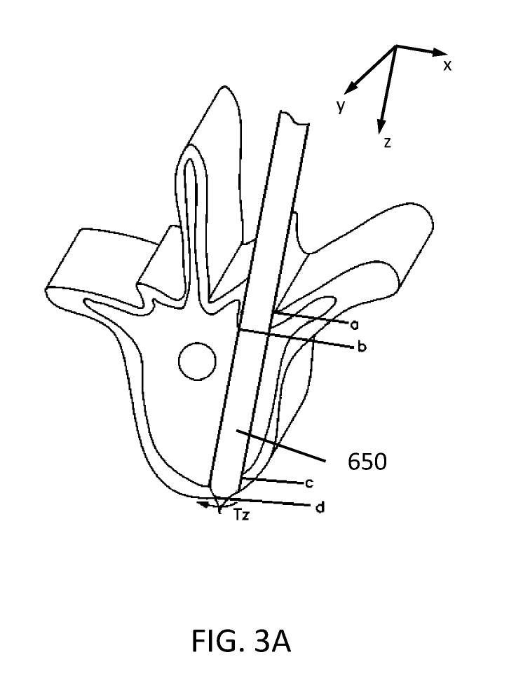

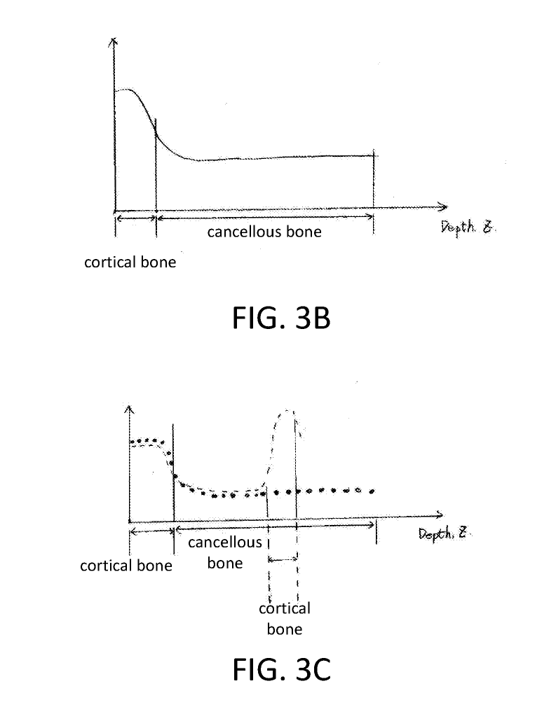

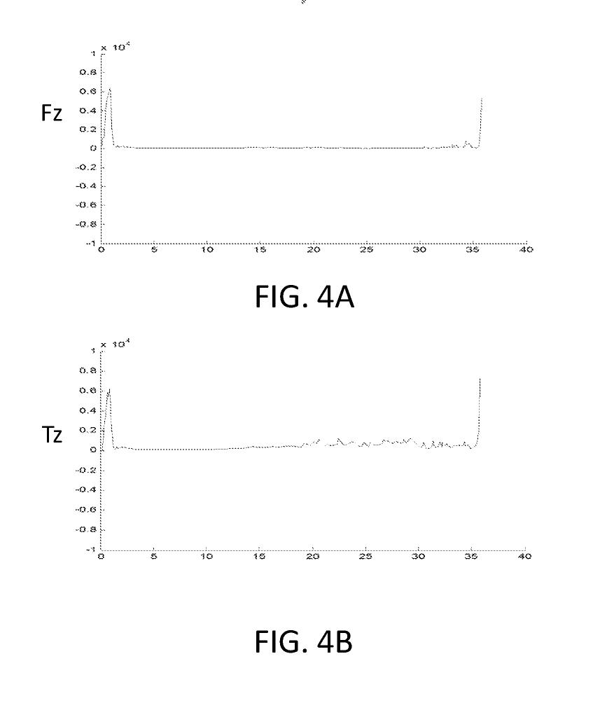

In one example as shown in FIG. 3A, an image information is reconstructed as a three-dimensional model from a series of CT images for spinal pedicle drilling process. In some examples, the biomechanical information may comprise of biomechanical properties along the planned drilling trajectory. Then the surgical tool 210 touches the entry point (denoted as a in FIG. 3A) of a vertebra. When the surgical tool starts breaking through the cortical bone on the vertebra, the value of the biomechanical property increases at the beginning and then drops to lower value after the tool penetrates the boundary (denoted as b in FIG. 3A) between the cortical bone and the cancellous bone. Afterwards, a different spindle speed, say a low spindle speed, is assigned to the drilling tool. The biomechanical property keeps low values until the tool touches another boundary (denoted as c in FIG. 3A) between the cortical bone and the cancellous bone again. At the exit point (denoted as d in FIG. 3A) of pedicle, the biomechanical property decease drastically.

As shown in FIG. 3B, the planning information comprises the spindle speed varying along the drilling depth. Different spindle speeds of the surgical tool are assigned for different stages in drilling process. The spindle speed profile of the drilling tool can be determined from the simulation of the surgical planning software. Drilling the cortical bone at a high spindle speed can reduce the possibility of deviation from the planned trajectory at this critical stage of bone drilling procedure. For example, a high spindle speed is assigned when the surgical tool touches the entry point of the cortical bone to achieve a desired feed rate along the planned drilling trajectory. After breaking through into the cancellous bone, the spindle speed is decreased by the control unit to have better detection of the biomechanical property. Therefore, the discrepancy index is more sensitive if the drilling information does not match the biomechanical information.

As shown in FIG. 3C, the biomechanical property along the drilling depth is distinguishable at a low spindle speed. The biomechanical properties for drilling cortical bone and cancellous bone at a low spindle speed can be more distinguishable than at a high spindle speed. During simulation, the control unit is also capable of generating the biomechanical information along other trajectories. At an optimized spindle speed, the surgical tool maintains good stability on the planned trajectory and the biomechanical properties of the planned trajectory and other fault trajectories are distinguishable to the control unit.

The biomechanical information comprising a biomechanical property per voxel is generated from the image information. The planning information comprising a planned drilling trajectory and a planned spindle speed may be predetermined by a surgeon or may be determined by optimization algorithm. In the example, the planned drilling trajectory is starting from the pedicle of a lumbar vertebra to the vertebral body. For ease of description, the z-axis is defined along the planned drilling trajectory, y-axis is defined as perpendicular to the vertebral body, and x-axis is the cross product of y-axis and z-axis. Accordingly, biomechanical information comprising biomechanical properties per voxel along the planned drilling trajectory can be predicted. The image information may be reconstructed into biomechanical information comprising biomechanical properties (denoted as u) and tissue types (denoted as t) corresponding to spatial location with three reference axes (denoted as rx, ry, rz). For example, each voxel with certain biomechanical information may be described as V(rx, ry, rz, t, u). In biomechanical simulation, the simulated force or torque may be calculated according to the cutting speed, uncut thickness, rake angle, inclination angle and width of the cutting edge element in each voxel under the condition of the planned information. The biomechanical property may be stored as a vector in directional components. For example, a z-component of the biomechanical property may be calculated as the torque along the z-axis divided by the planned spindle speed. In addition, the biomechanical property may be the force divided by the planned feed rate, the force divided by the planned spindle speed, or the torque divided by the feed rate. Tissue type may be classified according to the CT number (or Hounsfield unit) and may be used to highlight the neural tissue so that the control drilling system is capable of avoiding damage to the neural tissue. The planned drilling trajectory is determined before the drilling process by a surgeon or computer-assisted program.

The biomechanical information may be the biomechanical property as a function of drilling depth. One of the typical drilling impedance patterns, for example, may display the large value at the entry point, then drops to low values and last for a certain distance in the pedicle tunnel due to low resistance of the cancellous bone inside the pedicle. Afterwards the tool reaches the cortical bone at the exit of the pedicle, the drilling impedance again increases to high values at the contact of cortical bone and drops to low values after breaking through the cortical bone. However, if the tool deviates from the planned trajectory for some reasons, the increasing or dropping pattern of the drilling information will display earlier than expected location on the planned trajectory even though the image displays that tool is on the planned trajectory. The difference of drilling impedance pattern will be able to be used as a second opinion and gives a warning to the surgeon for safety check for the possibility of tool deviation.

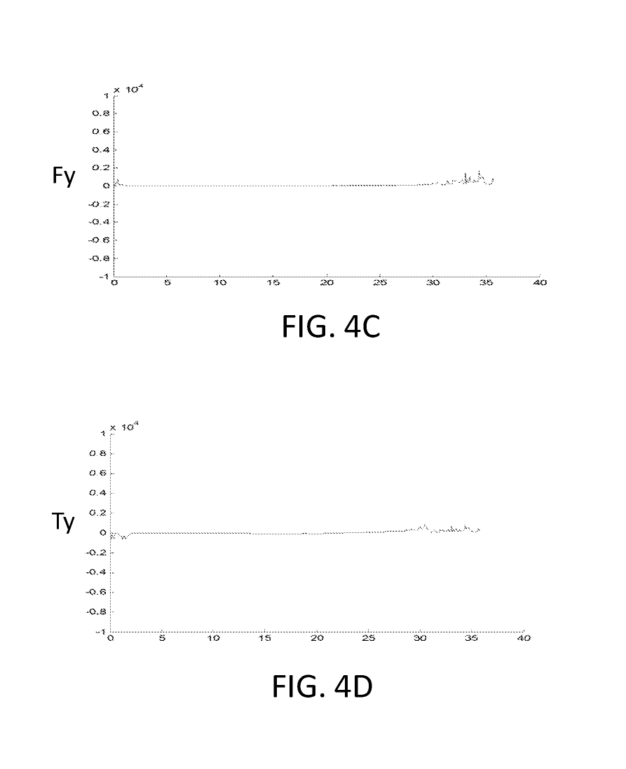

The biomechanical information may be simulated according to at least one force along an axis or one torque along an axis in varying drilling depth. As shown in FIG. 4A, the biomechanical property is simulated according to the force along z-axis. As shown in FIG. 4B, the biomechanical property is simulated according to the torque along the z-axis. As shown in FIG. 4C, the biomechanical property is simulated according to the force along y-axis. As shown in FIG. 4D, the biomechanical property is simulated according to the torque along y-axis. As shown in FIG. 4E, the biomechanical property is simulated according to the force along x-axis. As shown in FIG. 4F, the biomechanical property is simulated according to the torque along x-axis.

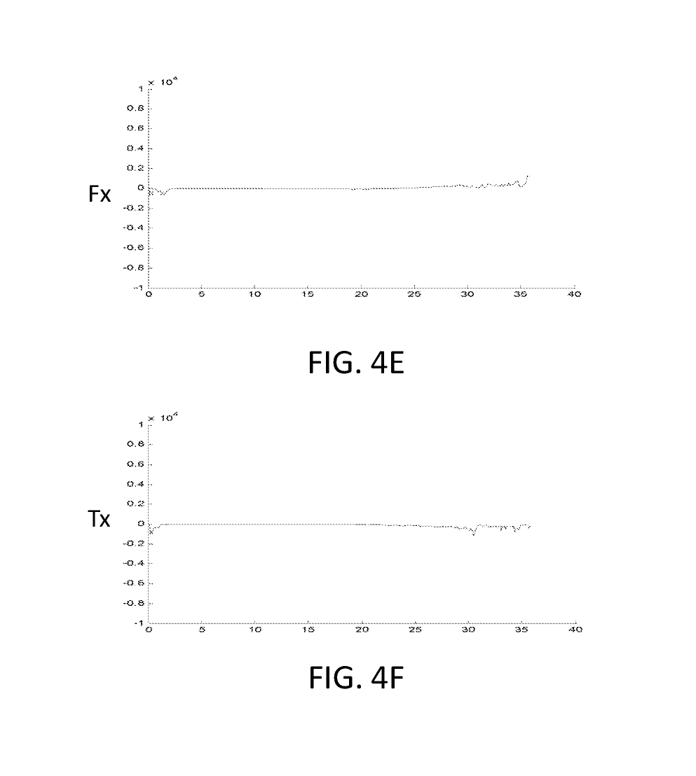

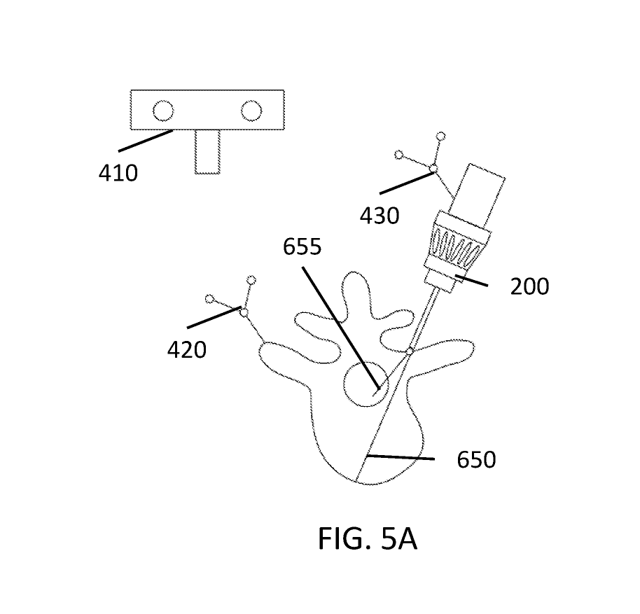

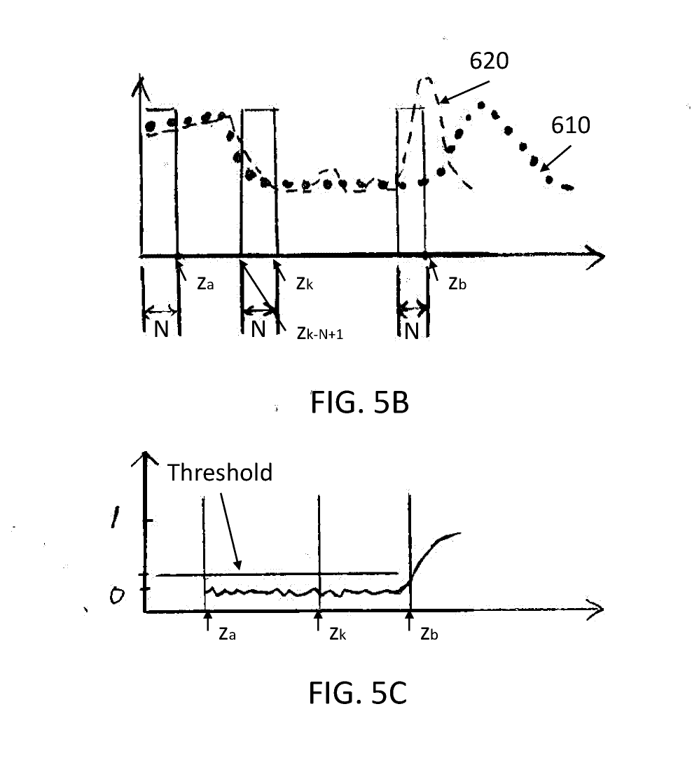

In one example as shown in FIG. 5A, the drilling control system is applied on a spinal pedicle drilling process. The mechanical sensor 220 detects mechanical information and the spatial sensor system detects the spatial information. In one example, the spatial sensor system acquires the spatial information by the tracking sensor 410 detecting the fiducial marker 420 and the device marker 430. The drilling information comprising the measured biomechanical property along the drilling trajectory will be compared with the biomechanical information comprising the biomechanical property along the planned trajectory. The differences of the drilling information and the biomechanical information are used for the judgment whether the surgical tool 210 is following the planned trajectory.

As shown in FIG. 5B, the biomechanical information 610 is presented as the biomechanical property under the condition of the planning information and the drilling information 620 is the measured biomechanical property recorded as a function of the spatial information. The measured biomechanical property is derived from the mechanical information, the spatial information and the spindle information. For example, the measured biomechanical property may be defined as the ratio of the force/torque over the surgical tool's feed rate/spindle speed along the moving direction. The control unit 600 monitoring the deviation between the drilling information and the biomechanical information.

In the example, the deviation may be determined by the discrepancy index. The discrepancy index is calculated according to the correlation between a first data window extracted from the biomechanical information 610 and a second data window extracted from the drilling information 620. First of all, a window with width N is assigned (as shown in FIG. 5B). The biomechanical information 610 is represented as the biomechanical property, Iₚ, as a function of the drilling depth z. The discrete calculation of the cross correlation between the biomechanical information and the drilling information in the window with width Nis presented as:

$${r}_{pm} ( {z}_{k} ) = {\sum }_{n = k - N + 1}^{k} {I}_{p} ( {z}_{n} ) {I}_{m} ( {z}_{n} ) ,$$

where zₖ is the kth sample of the drilling depth, n is the nth sample of the drilling depth, rₚₘ(zₖ) is the cross correlation of Ip and Im at drilling depth zₖ, (zₙ) is the biomechanical property at the nth sample of the drilling depth along the planned trajectory, and Iₘ(zₙ) is the measured biomechanical property at the nth sample of the drilling depth during the drilling process. Furthermore, the normalized cross correlation is calculated as:

$${ρ}_{pm} ( {z}_{k} ) = \frac{{r}_{pm} ( {z}_{k} )}{\sqrt{{r}_{pp} ( {z}_{k} ) {r}_{mm} ( {z}_{k} )}} , \\ where$$

$${r}_{pp} ( {z}_{k} ) = {\sum }_{n = k - N + 1}^{k} {I}_{p} ( {z}_{n} ) {I}_{p} ( {z}_{n} ) , \\ {r}_{mm} ( {z}_{k} ) = {\sum }_{n = k - N + 1}^{k} {I}_{m} ( {z}_{n} ) {I}_{m} ( {z}_{n} ) .$$

ρₚₘ(zₖ) is defined as the cross correlation normalized by the square root of the product of the autocorrelation. Then the discrepancy index is defined as: Ψ(zₖ)=1−ρₚₘ(zₖ). The discrepancy index is zero when these two curves are completely matched and increases from zero when one of the two curves is away from the other.

As shown in FIG. 5C, the discrepancy index along drilling depth is represented corresponding to the biomechanical information 610 and the drilling information 620 in FIG. 5B. During the depth from zₐ to zₖ, the discrepancy index is around zero. At the depth z_{b}, the drilling information 620 shows increasingly deviated from the biomechanical information 610. Therefore, the increase of the discrepancy index is noted. The control unit detects the discrepancy index and then send a control signal to slow or even stop the drilling motor if the discrepancy index is greater than the predetermined threshold.

In another example, the discrepancy index is calculated according to the slope of the biomechanical information and the slope of the drilling information. The control output is determined by the discrepancy index compared to a defined threshold. For example, the control output may be an alarm signal triggered or a spindle speed control signal to decrease the spindle speed when the discrepancy index is greater than the defined threshold; the control output may be a spindle speed control to keep the spindle rate when the discrepancy index is smaller than the defined threshold.

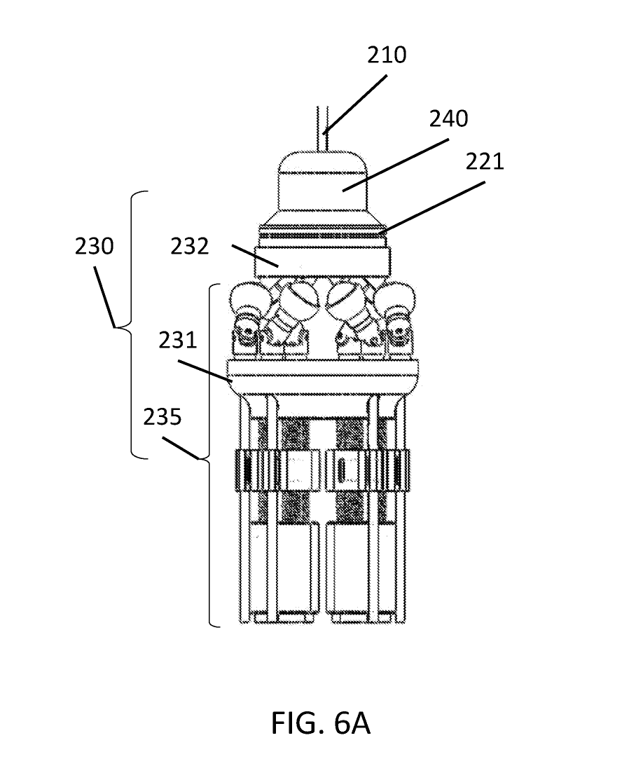

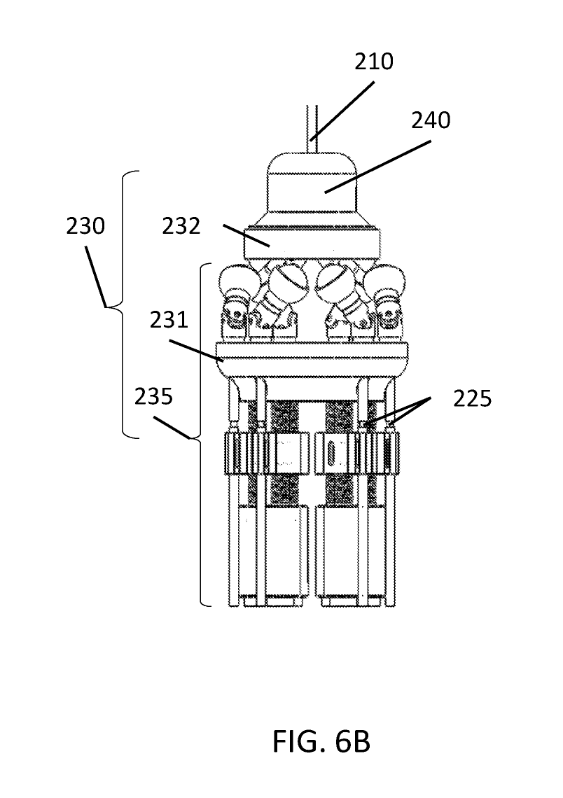

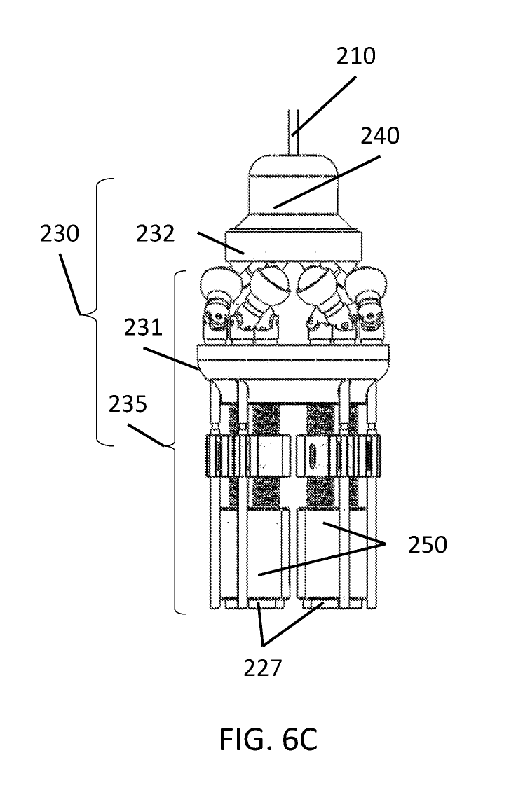

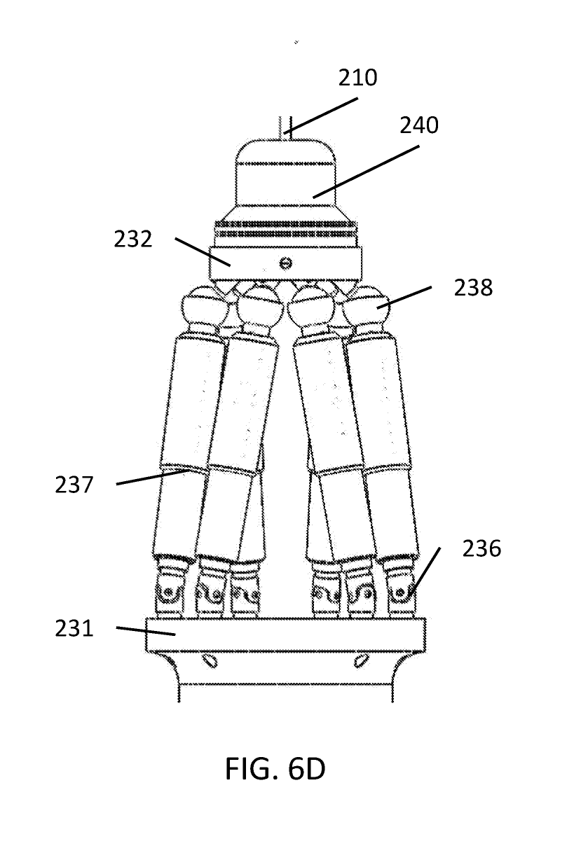

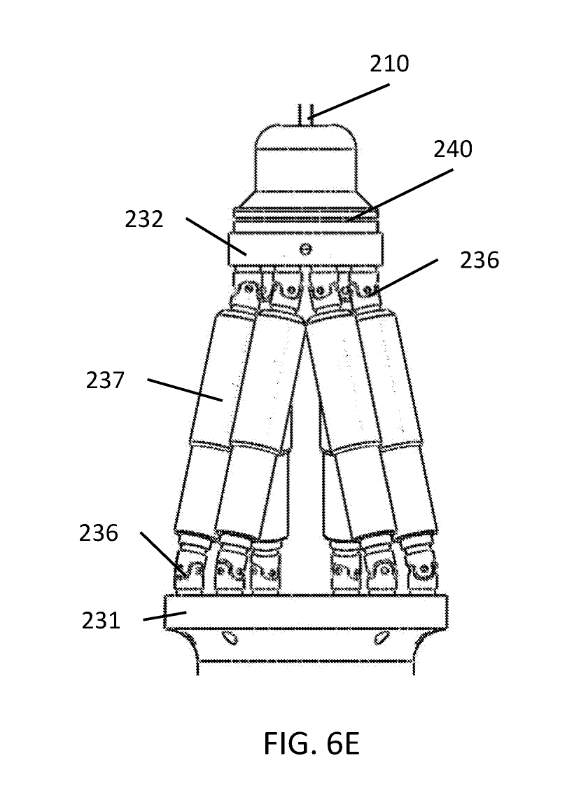

In one example as shown in FIG. 6A, the mechanical sensor is a force/torque sensor 221 capable of sensing the force in x-axis, y-axis, z-axis and the torque in x-axis, y-axis, z-axis. The mechanical sensor may be a six-axis force/torque sensor 221 coupled to the moving platform 232 of the robotic assembly 230 and the surgical tool 210, wherein the force/torque sensor 221 detects mechanical information including the force and the torque along x-axis, y-axis and z-axis and delivers the mechanical information to the control unit.

In one example as shown in FIG. 6B, the mechanical sensor may be a joint force sensor 225 capable of sensing the strain or the force along the kinetic pair. The joint force sensor 225 may be a strain gauge coupled to the kinetic pairs 235 of the robotic assembly, wherein the joint force sensor 225 detects mechanical information and delivers the mechanical information to the control unit. The joint force sensors 223 is capable of sensing the force and the torque along x-axis, y-axis and z-axis.

In one example as shown in FIG. 6C, the mechanical sensor is a motor current sensor coupled to the driving motors of the robotic assembly, wherein the mechanical sensor 220 detects mechanical information and delivers the mechanical information to the control unit. The drilling device may comprise multiple driving motors for the kinetic pairs and each of the motor current sensors is coupled to one driving motor of the robotic assembly. The mechanical sensor 220 is capable of sensing the electric current of the driving motors and then calculating the force and the torque along x-axis, y-axis and z-axis.

In one example as shown in FIG. 6D, the robotic assembly may be a Stewart type platform comprising six universal-prismatic-spherical (UPS) kinetic pairs. The UPS pair comprises a universal joint pair 236 coupled to the base platform 231, a prismatic joint pair 237 coupled to the universal joint pair 236 and a spherical joint pair 238 coupled to the moving platform 232 and the spherical joint pair 238.

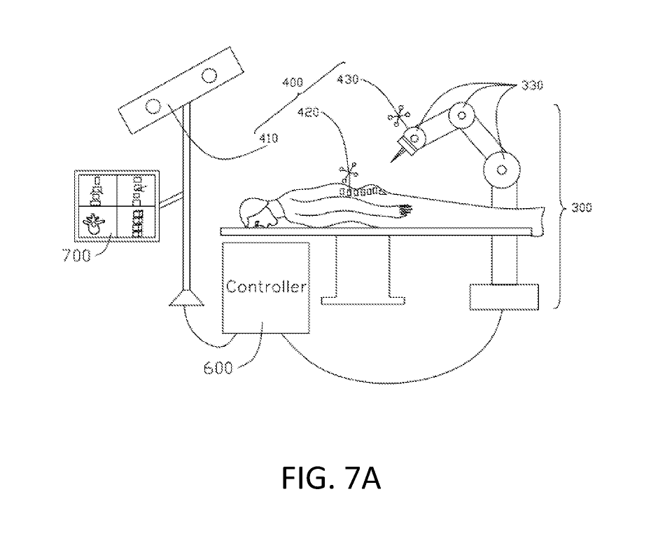

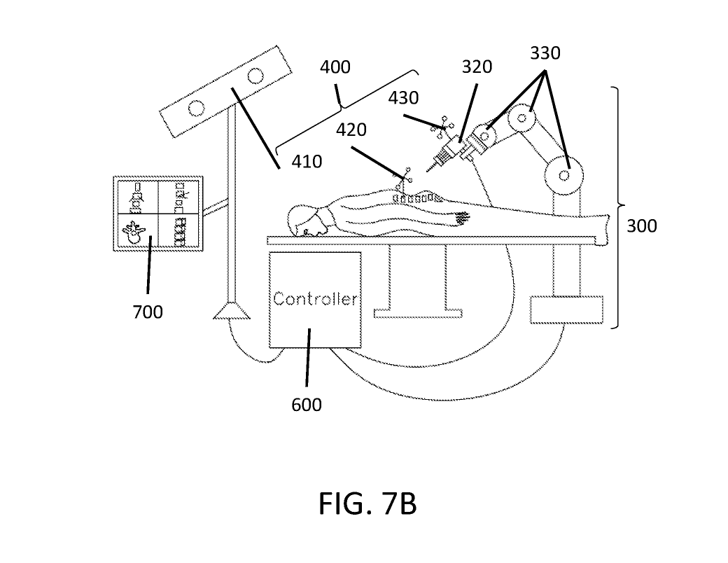

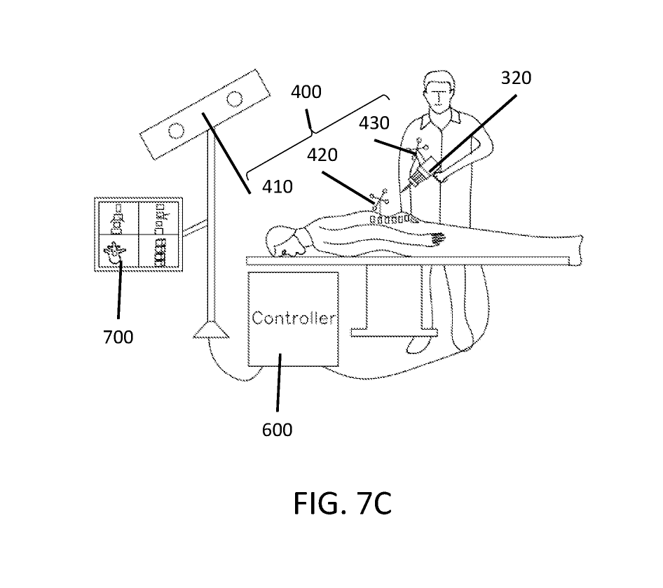

In one example as shown in FIG. 6E, the robotic assembly may be a Stewart type platform comprising six universal-prismatic-spherical (UPS) kinetic pairs. The UPS pair comprises a universal joint pair 236 coupled to the base platform 231, a prismatic joint pair 237 coupled to the universal joint pair 236 and a universal joint pair 236 coupled to the moving platform 232. In one example as shown in FIG. 7A, the drilling control system comprises an optical tracking system, a drilling device 200 and a control unit 600, wherein the operation base 300 of the drilling device 200 is a fixation base 310. The fixation base provides 310 mechanical stability so that the robotic assembly is steadily controlled with minimal unexpected movement. The fixation base 310 may be standing on the floor, hung on the ceiling or clamped to an operation table. The fixation base 310 may further comprise multiple mechanical joints 330 to stabilize the motion of the drilling device 200.

In one example as shown in FIG. 7B, the operation base 300 comprising the fixation base 310 may further comprise a handheld handle 320 and mechanical joints 330 so that the surgeon may have a degree of motion control of the drilling device 200.

In one example as shown in FIG. 7C, the fixation base 300 is a handheld handle 320 so that the surgeon may have most motion control of the drilling device 200 and compatible with the surgeon's user experience.

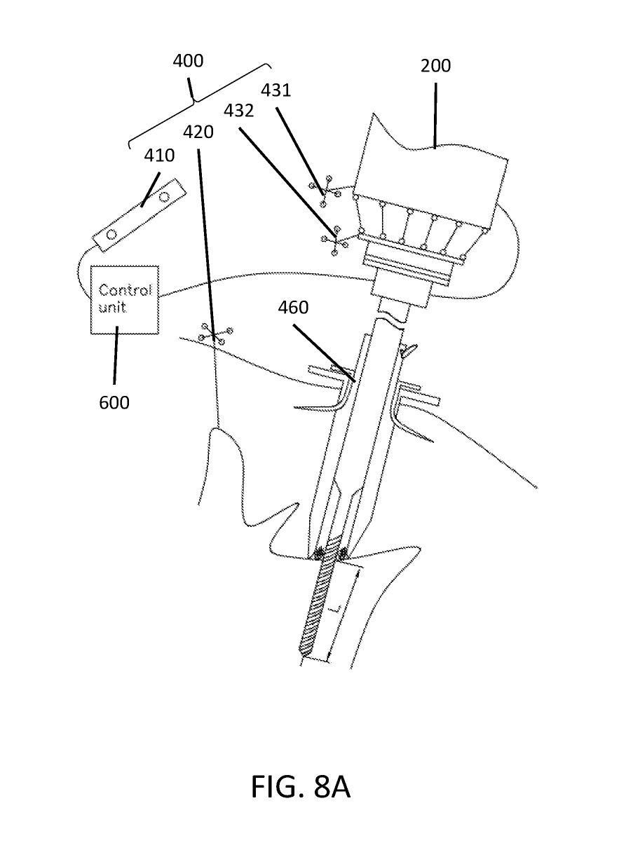

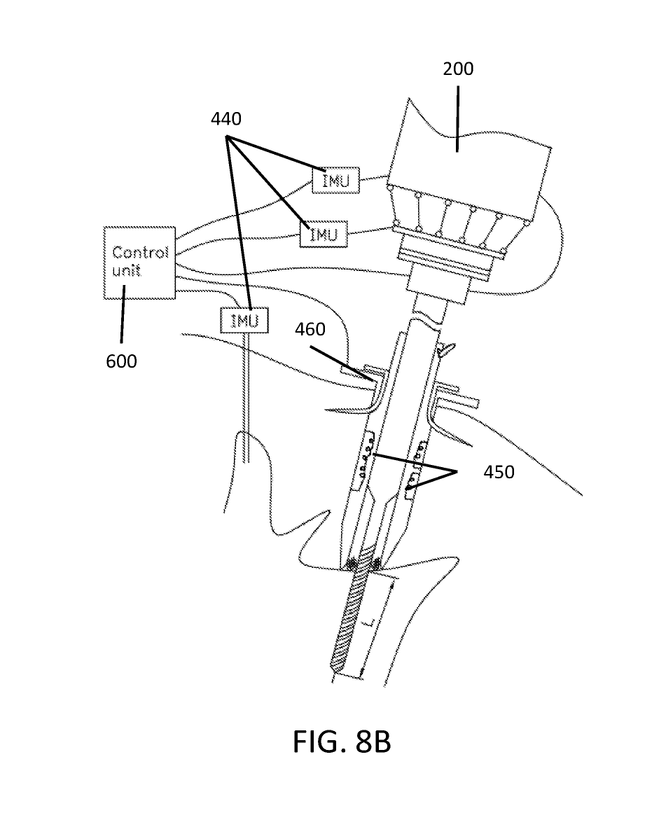

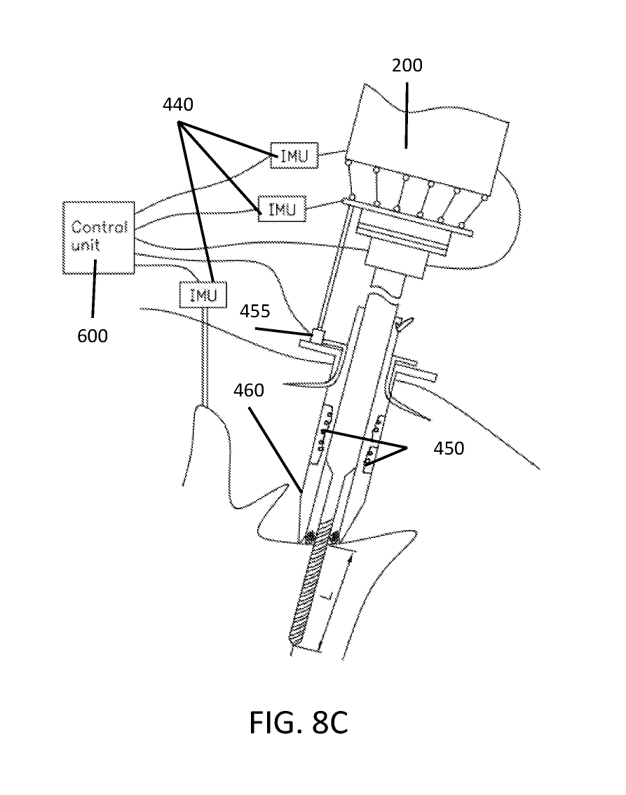

In one example as shown in FIG. 8A, the spatial sensor system 400 is a drilling trocar 460 comprising a position sensor 450, wherein the position sensor 450 detects the spatial information of the drilling device 200 and delivers the spatial information to the control unit 600. The position sensor 450may be configured on the tunnel of the trocar so that the spatial information comprising at least one degree of freedom as drilling depth is detected. Furthermore, the spatial sensor system 400 may be a combination of the drilling trocar and the optical tracking system capable of detecting spatial information comprising six degree of freedom.

In one example as shown in FIG. 8B, the spatial sensor system 400 is a drilling trocar 460 comprising a position sensor 450, wherein the position sensor 450 detects the spatial information of the drilling device 200 and delivers the spatial information to the control unit 600. The position sensor 450 may be configured on the tunnel of the drilling trocar 460 so that the spatial information comprising at least one degree of freedom as drilling depth is detected. The position sensor 450 may be a linear variable displacement transducer (LVDT) or a displacement sensor. Furthermore, the spatial sensor system 400 may be a combination of the drilling trocar and the inertial measurement units (IMU) 440 capable of detecting spatial information comprising six degree of freedom. In one example, the IMUs 440 may be configured on the base platform, moving platform 232 and an anatomical site.

In one example as shown in FIG. 8C, the spatial sensor system 400 is a drilling trocar 460 comprising a position sensor 450, wherein the position sensor 450 detects the spatial information of the drilling device 200 and delivers the spatial information to the control unit 600. The position sensor 450 may be configured on the outer part of the trocar so that the spatial information comprising at least one degree of freedom as drilling depth is detected. In the example, the position sensor may be a telemeter or a proximeter 455 to detect the distance between the outer part of the drilling trocar 460 and the moving platform 232. Furthermore, the spatial sensor system 400 may be a combination of the drilling trocar and the optical tracking system capable of detecting spatial information comprising six degree of freedom.

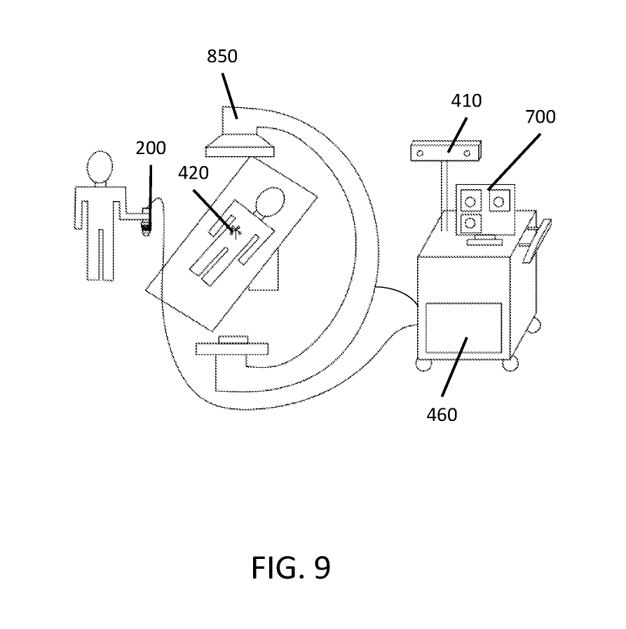

In one example as shown in FIG. 9, the drilling control system may receive the image information from a C-arm fluoroscopy to update the biomechanical information. Furthermore, the image information from the C-arm fluoroscopy may be used to confirm the spatial information. The drilling control system comprises a drilling device 200 and a control unit 600 and the control unit 600 is coupled to a C-arm fluoroscopy 850. In addition, the C-arm fluoroscopy may provide a part of spatial information for confirming the position and the orientation of the surgical tool. The drilling control system may further comprise a user interface 700 coupled to the control unit 600 to visualize the biomechanical information and the drilling information.

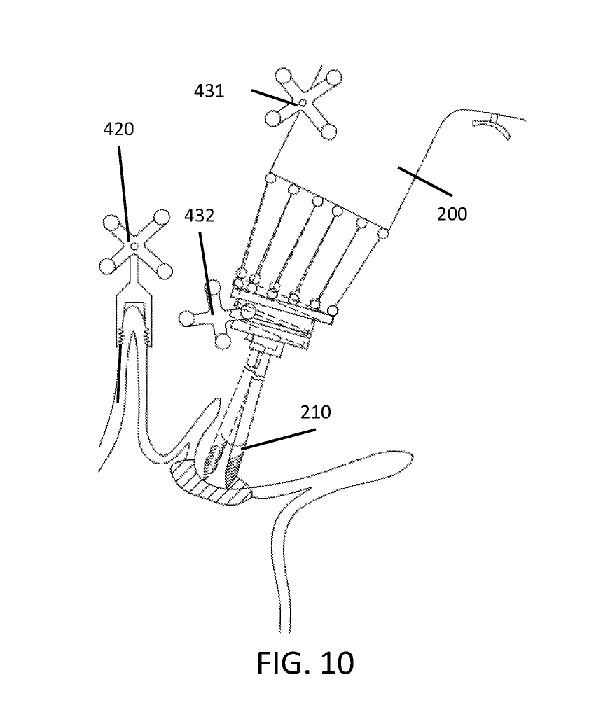

In one example as shown in FIG. 10, the robotic assembly may be a parallel manipulator configured to position the moving platform 232 with multi-degree-of-freedom. The control unit may generate control output according to the drilling information to compensate mis-alignment of the surgical tools during drilling process. Therefore the handheld robot-assisted surgical system can reduce the errors from surgeon's manual mis-alignment. When surgeon holds the handheld robot to the nearby of the target position/orientation on the vertebras, the handheld robot will automatically adjust the surgical tool 210 to the desired position/orientation and keep the desired position/orientation no matter any motion caused by surgeon's hand or anatomy. In one example as shown in FIG. 10, the control unit 600 may generate a control output according to the drilling information. The control output may be a motion control signal to control the robotic assembly or a spindle speed control signal to control the spindle rate of the drilling motor 240. The mechanical sensor 220 measures the forces and/or torques applied on the surgical tool 210 in the directions, for example, along x-axis, y-axis and z-axis. The robotic assembly adjusts the position/orientation of the surgical tool 210 according to the measured deviation forces/torques so that the deviation of the tool from the planned drilling trajectory can be reduced. Moreover, the force and/or torque along the planned trajectory together with the spatial information from marker and/or marker, is used to calculate the drilling impedance. Therefore, the robotic assembly can control the surgical tool 210 attached to the moving platform 232 to align with the desired position/orientation.

Furthermore, the control unit may send a motion control signal to the drilling device according to the planning information. For example, the planning information is the feed rate of drilling process. The drilling device may adjust the force apply on the z-axis by slightly protracting or retracting the robotic assembly. In addition, the drilling device may also be adjusted according to the force or the torque in x-axis and y-axis to reduce deviation from the planned drilling trajectory.

It is contemplated that the control unit may be a solitary work station coupled to the drilling device or may be a system in package embedded in the drilling device.

The examples shown and described above are only examples. Therefore, many such details are neither shown nor described. Even though numerous characteristics and advantages of the present technology have been set forth in the foregoing description, together with details of the structure and function of the present disclosure, the disclosure is illustrative only, and changes may be made in the detail, including in matters of shape, size and arrangement of the parts within the principles of the present disclosure up to, and including the full extent established by the broad general meaning of the terms used in the claims. It will therefore be appreciated that the examples described above may be modified within the scope of the claims.

What is claimed is:

1. A drilling control system comprising:
a drilling device comprising a surgical tool, a drilling motor driving the surgical tool, a mechanical sensor detecting mechanical information, a robotic arm assembly receiving a control output and detecting spindle information, and an operation base coupled to the robotic arm assembly;
a control unit coupled to the drilling device and a spatial sensor system, wherein the control unit stores biomechanical information, generates drilling information according to the mechanical information received from the mechanical sensor, the spindle information from the drilling device and spatial information received from the spatial sensor system, calculates a discrepancy index according to the biomechanical information and the drilling information, and sends the control output to the drilling device according to the discrepancy index.

2. The drilling control system of claim 1, wherein the discrepancy index is calculated according to cross correlation of the biomechanical information and the drilling information.

3. The drilling control system of claim 1, wherein the discrepancy index is calculated according to cross correlation of the slopes of the biomechanical information and the drilling information.

4. The drilling control system of claim 1, wherein the control output is an alarm signal.

5. The drilling control system of claim 1, wherein the control output is a spindle speed control signal.

6. The drilling control system of claim 1, wherein the control output is a motion control signal.

7. The drilling control system of claim 1, wherein the mechanical sensor is a force/torque sensor coupled to the drilling motor.

8. The drilling control system of claim 1, wherein the mechanical sensor is a joint force sensor coupled to the robotic assembly.

9. The drilling control system of claim 1, wherein the mechanical sensor is a motor current sensor coupled to the robotic assembly.

10. The drilling control system of claim 1, wherein the robotic assembly is a parallel manipulator.

11. The drilling control system of claim 7, wherein the robotic assembly is a Stewart type platform.

12. A drilling control method performed at a drilling control system, the method comprising:
detecting mechanical information at a mechanical sensor
receiving and storing, at the control unit, biomechanical information, mechanical information, spatial information and spindle information;
generating, at the control unit, drilling information according to the mechanical information the spatial information and the spindle information;
calculating, at the control unit, a discrepancy index according to the biomechanical information and the drilling information; and
sending, at the control unit, a control output to a drilling device according to the discrepancy index.

13. The drilling control method of claim 12, wherein the discrepancy index is calculated according to cross correlation of the biomechanical information and the drilling information.

14. The drilling control method of claim 12, wherein the discrepancy index is calculated according to cross correlation of the slopes of the biomechanical information and the drilling information.
